(19) United States
(12) Patent Application Publication
(10) Pub. No.: US 2024/0130683 A1
(43) Pub. Date: Apr. 25, 2024

(54) CATHETER CONTROL SYSTEM

(71) Applicants: THE TEXAS A&M UNIVERSITY SYSTEM, College Station, TX (US); THE METHODIST HOSPITAL SYSTEM, Houston, TX (US)

(72) Inventors: Debjyoti BANERJEE, College Station, TX (US); C. Huie LIN, Houston, TX (US)

(21) Appl. No.: 18/278,994

(22) PCT Filed: Feb. 25, 2022
(86) PCT No.: PCT/US2022/017906

§ 371 Date: Aug. 24, 2023

Related U.S. Application Data
(60) Provisional application No. 63/154,233, filed on Feb. 26, 2021.

(51) Int. Cl. A61B 5/00; A61M 25/00; A61M 25/01

(52) U.S. Cl. CPC A61B 5/6852; A61M 25/0023; A61M 25/0122; A61M 2025/0166

(57) ABSTRACT

A catheter control system may include a containment structure, a sensor coupled to the containment structure, and a plurality of actuators coupled to the containment structure. The containment structure may define a lumen configured to allow a fluid to flow therethrough, and a plurality of openings in fluid communication with the lumen. The sensor may be configured to detect a current position and/or orientation of the containment structure. Each of the actuators may be associated with at least one respective opening of the plurality of openings and configured to regulate fluid flow through the at least one respective opening.

### CROSS-REFERENCE TO RELATED APPLICATIONS

This application claims priority to and the benefit of U.S. Provisional Patent Application No. 63/154,233, filed on Feb. 26, 2021, the disclosure of which is expressly incorporated herein by reference in its entirety.

### FIELD OF THE DISCLOSURE

The present disclosure relates generally to catheters for insertion at least partially within the body of a subject (i.e., a human or an animal) and more particularly to catheter control systems for autonomously controlling a position and/or orientation of a catheter within a subject's body.

### BACKGROUND OF THE DISCLOSURE

Various types of medical devices may be inserted at least partially into a subject's body to supplement or correct natural bodily functions without invasive surgery. Such medical devices may include a catheter configured to allow a fluid, such as blood or another bodily fluid, to flow through a lumen of the catheter, and a pump configured to induce fluid flow through the lumen. Often, a particular position and/or orientation (i.e., attitude) of a catheter mounted device, such as a ventricular assist device, may maximize performance of the device. In some instances, the induced fluid flow undesirably may cause the catheter to move away from a target position (i.e., an intended position) and/or a target orientation (i.e., an intended orientation) within the subject's body, which may result in harm to the subject and/or may inhibit proper function of the medical device. In particular, the catheter may migrate away from the target position and/or target orientation due to inertial reactive forces generated by the fluid flow through the lumen. In some instances, the catheter may move away from the target position and/or target orientation due to movement of the subject's body, such as when the subject engages in physical activity.

As an example, mechanical circulatory support devices may be used for patients diagnosed with advanced heart failure to increase blood circulation and assist in heart function, thereby extending survival and improving quality of life. In contrast to surgically implanted mechanical circulatory support devices, percutaneous ventricular assist devices (PVADs) may allow for rapid deployment of temporary, partial circulatory support in a manner that does not require open heart surgery. A PVAD typically may rely on placement of a catheter at least partially inside the subject's heart, allowing blood to be drawn from the ventricle and pumped to the aorta via an external pump or an integrated impeller pump device positioned within the catheter. During use of the PVAD, the reactive forces generated by the flow of blood through the catheter or physical activity by the patient may cause the PVAD catheter to migrate away from its target position and/or target orientation. In some instances, such changes in the position and/or orientation of the PVAD catheter may be potentially catastrophic for the patient. For example, migration of the catheter may cause hemolysis, or mechanical shearing of red blood cells, not only causing complications similar to large volume blood loss but also inducing kidney and other end-organ injury due to release of massive amounts of hemoglobin and other intracellular contents into the bloodstream. Because of these potential concerns, identifying and maintaining the position as well as orientation of the PVAD catheter may be of critical importance. Currently, the position and/or orientation of the PVAD may be monitored with chest x-rays performed on a daily basis, and the catheter may be blindly repositioned and/or reoriented at the patient's bedside with a repeat x-ray to confirm the change in position and/or orientation. Alternatively, echocardiography, although often limited in image quality, may be used to visualize the catheter and aid in efforts to manually reposition and/or reorient the PVAD. Notably, these current methods are limited with respect to precision in identifying the PVAD position and/or orientation within the context of cardiac structures as well as precision in adjusting the PVAD position and/or orientation. Similar techniques may be used for determining the position and/or orientation of other types of medical devices within a subject's body and manually moving such devices toward a desired position and/or orientation, which may be cumbersome for the patient and physician.

A need therefore exists for improved medical devices having a catheter, such as PVADs, and related methods for controlling a position and/or orientation of a catheter within a subject's body, which may overcome one or more of the above-mentioned limitations associated with existing techniques for identifying and changing or maintaining a position and/or orientation of a catheter of a medical device within a subject's body.

### SUMMARY OF THE DISCLOSURE

The present disclosure provides catheter control systems and related methods for using such systems to navigate, monitor, position, and/or orient catheters within a subject's body. In one aspect, a catheter control system is provided. In one embodiment, the catheter position control system may include a containment structure, a sensor, and a plurality of actuators. The containment structure may define a lumen and a plurality of openings. The lumen may be configured to allow a fluid to flow therethrough. The plurality of openings may be in fluid communication with the lumen. The sensor may be coupled to the containment structure. The sensor may be configured to detect a current position and/or attitude of the containment structure. The plurality of actuators may be coupled to the containment structure. Each of the actuators may be associated with at least one respective opening of the plurality of openings and configured to regulate fluid flow through the at least one respective opening.

In some embodiments, the containment structure may have a tubular shape. In some embodiments, the containment structure may be flexible. In some embodiments, the containment structure may be rigid. In some embodiments, the containment structure may have a first end and a second end positioned opposite the first end, and the lumen may extend from the first end to the second end. In some embodiments, the plurality of openings may include a plurality of inlet openings configured to allow fluid to enter the lumen, each of the inlet openings may be positioned closer to the first end than the second end; and a plurality of outlet openings configured to allow fluid to exit the lumen, each of the outlet openings may be positioned closer to the second end than the first end. In some embodiments, the inlet openings may be positioned at the first end, and the outlet openings may be positioned at the second end. In some embodiments, the inlet openings may be equally spaced apart from one another, and the outlet openings may be equally spaced apart from one another. In some embodiments, the inlet openings may be oriented in a plurality of different directions from one another relative to the containment structure, and the outlet openings may be oriented in a plurality of different directions from one another relative to the containment structure. In some embodiments, the plurality of inlet openings may include a pair of inlet openings oriented in opposite directions from one another relative to the containment structure, and the plurality of outlet openings may include a pair of outlet openings oriented in opposite directions from one another relative to the containment structure. In some embodiments, the plurality of inlet openings may include at least two inlet openings, and the plurality of outlet openings may include at least two outlet openings. In some embodiments, the plurality of inlet openings may include at least three inlet openings, and the plurality of outlet openings may include at least three outlet openings. In some embodiments, the plurality of inlet openings may include at least six inlet openings, and the plurality of outlet openings may include at least six outlet openings. In some embodiments, a number of the inlet openings may be equal to a number of the outlet openings. In some embodiments, a number of the inlet openings may be greater than a number of the outlet openings. In some embodiments, a number of the inlet openings may be less than a number of the outlet openings.

In some embodiments, the containment structure may include a bellows configured to allow a length of the containment structure to be extended or retracted. In some embodiments, the catheter position control system may include a bellows actuator configured to extend or retract the bellows.

In some embodiments, the sensor may include an accelerometer. In some embodiments, the sensor may include a gyroscope. In some embodiments, the sensor may include an array of capacitive sensors. In some embodiments, the sensor may include an inductive sensor. In some embodiments, the sensor may include a magnetic sensor. In some embodiments, the sensor may include an optical sensor. In some embodiments, the sensor may include a piezoelectric sensor. In some embodiments, the plurality of openings may include an inlet opening configured to allow fluid to enter the lumen and an outlet opening configured to allow fluid to exit the lumen, and the sensor may be positioned closer to the inlet opening than the outlet opening. In some embodiments, the sensor may be positioned on an external surface of the containment structure. In some embodiments, the sensor may be positioned on an internal surface of the containment structure.

In some embodiments, each of the actuators may be configured to transition between a fully open state, a partially open state, and a closed state for regulating fluid flow through the at least one respective opening. In some embodiments, the plurality of actuators may include a plurality of valves. In some embodiments, the plurality of actuators may include a plurality of diaphragms. In some embodiments, the plurality of actuators may include a plurality of venturi devices.

In some embodiments, the catheter control system may include a pump in fluid communication with the lumen. In some embodiments, the pump may be configured to create a pressure differential for inducing fluid flow through the lumen. In some embodiments, the pump may be positioned at least partially within the containment structure. In some embodiments, the pump may be a screw pump.

In some embodiments, the catheter control system may include a pump actuator configured to drive the pump. In some embodiments, the pump actuator may include a pump motor. In some embodiments, the pump actuator may include a thermally driven actuator. In some embodiments, the pump actuator may include a piezoelectric actuator. In some embodiments, the pump actuator may be positioned at least partially within the containment structure. In some embodiments, the catheter control system may include a power source configured to power the pump actuator. In some embodiments, the power source may be positioned at least partially within the containment structure. In some embodiments, the power source may include a battery.

In some embodiments, the catheter control system may include an electronic controller in operable communication with the sensor and the actuators. In some embodiments, the electronic controller may be configured to cause one or more of the actuators to vary fluid flow through one or more of the openings based at least in part on the current position and/or orientation and a target position and/or orientation of the containment structure. In some embodiments, the electronic controller may be configured to cause one or more of the actuators to vary fluid flow through one or more of the openings proportional to a difference between the current position and/or orientation and the target position and/or orientation. In some embodiments, the electronic controller may be configured to cause one or more of the actuators to vary fluid flow through one or more of the openings based at least in part on a proportional function, a differential function, an integral function, a derivative function, or a combination thereof. In some embodiments, the electronic controller may be configured to cause one or more of the actuators to vary fluid flow through one or more of the openings based at least in part on a machine learning algorithm or a neural network learning algorithm. In some embodiments, the electronic controller may be configured to cause each of the actuators to transition between a fully open state, a partially open state, and a closed state for regulating fluid flow through the at least one respective opening.

In another aspect, a method for controlling a position and/or orientation of a catheter is provided. In one embodiment, the catheter may have a containment structure defining a lumen and a plurality of openings in fluid communication with the lumen, and the method may include: determining, via a sensor coupled to the containment structure, a current position and/or orientation of the containment structure; and causing, via one or more actuators coupled to the containment structure, fluid flow through one or more of the openings to be varied based at least in part on the current position and/or orientation and a target position and/or orientation of the containment structure such that the containment structure moves from the current position and/or orientation toward the target position and/or orientation.

In some embodiments, the containment structure may have a tubular shape. In some embodiments, the containment structure may be flexible. In some embodiments, the containment structure may be rigid. In some embodiments, the containment structure may have a first end and a second end positioned opposite the first end, and the lumen may extend from the first end to the second end. In some embodiments, the plurality of openings may include a plurality of inlet openings configured to allow fluid to enter the lumen, and a plurality of outlet openings configured to allow fluid to exit the lumen. Each of the inlet openings may be positioned closer to the first end than the second end, and each of the outlet openings may be positioned closer to the second end than the first end. In some embodiments, the inlet openings may be positioned at the first end, and the outlet openings may be positioned at the second end. In some embodiments, the inlet openings may be equally spaced apart from one another, and the outlet openings may be equally spaced apart from one another. In some embodiments, the inlet openings may be oriented in a plurality of different directions from one another relative to the containment structure, and the outlet openings may be oriented in a plurality of different directions from one another relative to the containment structure. In some embodiments, the plurality of inlet openings may include a pair of inlet openings oriented in opposite directions from one another relative to the containment structure, and the plurality of outlet openings may include a pair of outlet openings oriented in opposite directions from one another relative to the containment structure. In some embodiments, the plurality of inlet openings may include at least two inlet openings, and the plurality of outlet openings may include at least two outlet openings. In some embodiments, the plurality of inlet openings may include at least three inlet openings, and the plurality of outlet openings may include at least three outlet openings. In some embodiments, the plurality of inlet openings may include at least six inlet openings, and the plurality of outlet openings may include at least six outlet openings. In some embodiments, a number of the inlet openings may be equal to a number of the outlet openings. In some embodiments, a number of the inlet openings may be greater than a number of the outlet openings. In some embodiments, a number of the inlet openings may be less than a number of the outlet openings.

In some embodiments, the containment structure may include a bellows configured to allow a length of the containment structure to be extended or retracted. In some embodiments, the method may include extending or retracting the bellows via a bellows actuator.

In some embodiments, the sensor may include an accelerometer. In some embodiments, the sensor may include a gyroscope. In some embodiments, the sensor may include an array of capacitive sensors. In some embodiments, the sensor may include an inductive sensor. In some embodiments, the sensor may include a magnetic sensor. In some embodiments, the sensor may include an optical sensor. In some embodiments, the sensor may include a piezoelectric sensor. In some embodiments, the plurality of openings may include an inlet opening configured to allow fluid to enter the lumen and an outlet opening configured to allow fluid to exit the lumen, and the sensor may be positioned closer to the inlet opening than the outlet opening. In some embodiments, the sensor may be positioned on an external surface of the containment structure. In some embodiments, the sensor may be positioned on an internal surface of the containment structure.

In some embodiments, determining the current position of the containment structure may include integrating an output signal of the sensor as a function of time. In some embodiments, the output signal may be indicative of one or more of: (i) linear acceleration of the containment structure, (ii) angular acceleration of the containment structure, (iii) linear velocity of the containment structure, and (iv) angular velocity of the containment structure.

In some embodiments, causing fluid flow through the one or more of the openings to be varied may include causing the one or more of the actuators to transition between a fully open state, a partially open state, and a closed state. In some embodiments, the plurality of actuators may include a plurality of valves. In some embodiments, the plurality of actuators may include a plurality of diaphragms. In some embodiments, the plurality of actuators may include a plurality of venturi devices.

In some embodiments, the method may include causing, via a pump, a pressure differential to be created for inducing fluid flow through the lumen. In some embodiments, the pump may be positioned at least partially within the containment structure. In some embodiments, the pump may be a screw pump. In some embodiments, the method may include causing the pump to be driven via a pump actuator. In some embodiments, the pump actuator may include a pump motor. In some embodiments, the pump actuator may include a thermally driven actuator. In some embodiments, the pump actuator may include a piezoelectric actuator. In some embodiments, the pump actuator may be positioned at least partially within the containment structure. In some embodiments, the method may include causing the pump actuator to be powered via a power source. In some embodiments, the power source may be positioned at least partially within the containment structure. In some embodiments, the power source may include a battery.

In some embodiments, causing fluid flow through the one or more openings to be varied may include causing, via a controller, the one or more actuators to vary fluid flow through the one or more of openings proportional to a difference between the current position and/or orientation and the target position and/or orientation. In some embodiments, causing fluid flow through the one or more openings to be varied may include causing, via a controller, the one or more actuators to vary fluid flow through the one or more of openings based at least in part on a proportional function, a differential function, an integral function, a derivative function, or a combination thereof. In some embodiments, causing fluid flow through the one or more openings to be varied may include causing, via a controller, the one or more actuators to vary fluid flow through the one or more of openings based at least in part on a machine learning algorithm or a neural network learning algorithm. In some embodiments, causing fluid flow through the one or more openings to be varied may include causing, via a controller, the one or more actuators to transition between a fully open state, a partially open state, and a closed state. In some embodiments, the controller may include an electronic controller. In some embodiments, the controller may include a mechanical controller. In some embodiments, the controller may include a thermal controller. In some embodiments, the controller may include a piezoelectric controller. In some embodiments, the controller may include an acoustic controller.

These and other aspects and improvements of the present disclosure will become apparent to one of ordinary skill in the art upon review of the following detailed description when taken in conjunction with the several drawings and the appended claims.

### BRIEF DESCRIPTION OF THE DRAWINGS

FIG. 1 schematically illustrates an example catheter control system in accordance with one or more embodiments of the disclosure, showing a containment structure, a sensor, a plurality of actuators, a pump, a pump motor, a power source, and a controller of the catheter control system.

FIGS. 2A-2B schematically illustrate the catheter control system of FIG. 1 integrated with an example PVAD.

FIGS. 3A-3B schematically illustrate the catheter control system of FIG. 1 integrated with another example PVAD.

FIG. 4 schematically illustrates a method for controlling a position and/or orientation of a catheter within a subject's body in accordance with one or more embodiments of the disclosure, showing a closed-loop feedback control scheme.

FIGS. 5A-5H illustrate example data from experimental testing using an accelerometer to determine position.

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. The use of the same reference numerals indicates similar, but not necessarily the same or identical components. Different reference numerals may be used to identify similar components. Various embodiments may utilize elements or components other than those illustrated in the drawings, and some elements and/or components may not be present in various embodiments. The use of singular terminology to describe a component or element may, depending on the context, encompass a plural number of such components or elements and vice versa.

### DETAILED DESCRIPTION OF THE DISCLOSURE

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

Embodiments of catheter control systems and related methods of using such systems for controlling a position and/or orientation of a catheter within the body of a subject (i.e., a human or an animal) are provided. The catheter control system may be implemented with or as a part of a medical device configured for insertion at least partially within a subject's body. For example, the catheter control system may be attached to or integrated with a mechanical circulatory support device, such as a PVAD, although one skilled in the art will appreciate that the catheter control system may be used in a similar manner with other types of medical devices having a catheter for fluid flow. As described herein, the catheter control system may be provided as an integrated system that combines spatial position and/or orientation monitoring and sensing with actuation devices that use feedback control strategies for position and/or attitude control of the catheter. The catheter control system may precisely determine and monitor the real-time position and/or attitude of the catheter with respect to the subject's anatomy during and after interventional procedures via an inertial navigation system (e.g., miniaturized accelerometers or gyroscopes). Miniaturization schemes may utilize MEMS (Micro-Electro-Mechanical-Systems) techniques, such as MEMS capacitors or accelerometers, in combination with gyroscopes. For example, sensor signals (e.g., linear and angular acceleration or velocity) may be integrated as a function of time, along the three Cartesian coordinate directions, such that the position and/or orientation of the catheter may be accurately determined and monitored with respect to a specified datum or reference plane. In addition to accurately detecting the real-time position and/or orientation of the catheter, the catheter control system may provide precise, real-time control of the catheter position and/or orientation in three-dimensional space. The catheter control system may autonomously control the catheter position and/or orientation based at least in part on a current position and/or orientation (i.e., a real-time position and/or orientation) of the catheter and a target position and/or orientation (i.e., a desired position and/or orientation) of the catheter. In particular, one or more actuators of the catheter control system may be used to regulate fluid flow through one or more openings of the catheter such that the reactive forces generated by the fluid flow result in a propulsive thrust for moving the catheter from the current position and/or orientation to, or at least toward, the target position and/or orientation.

As described herein, a catheter control system generally may include a containment structure that defines a lumen configured to allow a fluid to flow therethrough and a plurality of openings in fluid communication with the lumen, a sensor that is coupled to the containment structure and configured to detect a current position and/or attitude of the containment structure, and a plurality of actuators that are coupled to the containment structure. Each of the actuators may be associated with at least one respective opening of the plurality of openings and configured to regulate fluid flow through the at least one respective opening. As described below, the actuators may be used to regulate the fluid flow through the respective openings based on real-time position and/or orientation feedback from the sensor. The catheter control system may include a controller that is in operable communication with the sensor and the actuators. In this manner, the controller may receive a sensor signal from the sensor and then may send a control signal to one or more of the actuators for regulating fluid flow through one or more of the openings to move the containment structure from the current position to, or at least toward, a target position. Similarly, the controller may receive a sensor signal from the sensor and then may send a control signal to one or more of the actuators for regulating fluid flow through one or more of the openings to move the containment structure from the current angular orientation to, or at least toward, a target orientation (or a combination of orientation and position). For example, when the controller determines, based at least in part on the sensor signal, that the current position and/or orientation of the containment structure is different from the target position and/or orientation, the controller may cause the actuators to regulate the fluid flow through the respective openings such that the reactive forces created by the fluid flow reposition and/or reorient the containment structure to, or at least toward, the target position and/or orientation. The plurality of openings may include a plurality of inlet openings configured to allow fluid to enter the lumen, and a plurality of outlet openings configured to allow fluid to exit the lumen. The inlet openings and the outlet openings may be oriented in a plurality of different directions from one another relative to the containment structure such that the containment structure may be repositioned and reoriented relative to multiple fixed axes. In some embodiments, the catheter control system may be attached to an existing medical device, such as an existing PVAD or other type of mechanical circulatory support device, that is configured for insertion into a subject's body, such that the position and/or attitude of the medical device within the body may be tracked and controlled. In some embodiments, the catheter control system may be integrated into a medical device, such as a PVAD or other type of mechanical circulatory support device, that is configured for insertion into a subject's body, such that the position and/or attitude of the medical device within the body may be tracked and controlled. In such embodiments, any pumps or actuators associated with the medical device may be used in combination with the catheter control system to further regulate fluid flow for positioning and orienting the medical device.

As discussed above, existing techniques for identifying and changing or maintaining a position and/or orientation of a catheter of a medical device within a subject's body may have certain limitations. In some instances, fluid flow through a lumen of the catheter may cause the catheter to move away from a target position and/or orientation within the subject's body, which may result in harm to the subject and/or may inhibit proper function of the medical device. Specifically, the catheter may migrate away from the target position and/or orientation due to inertial reactive forces generated by the fluid flow through the lumen. In some instances, the catheter may move away from the target position and/or orientation due to movement of the subject's body, such as when the subject engages in physical activity. In the context of certain mechanical circulatory support devices, such as PVADs, and other similar types of medical devices inserted at least partially into a subject's heart, reactive forces caused by induced blood flow and/or physical movement of the subject may cause the catheter to migrate away from its target position and/or orientation, resulting in complications, such as hemolysis. Current approaches, which rely on use of x-rays or echocardiography to determine a position and/or orientation of a catheter of a medical device, such as a PVAD, and manual repositioning and/or reorienting of the catheter, generally may lack the degree of precision desired in many applications and may be cumbersome for the subject and the physician.

The catheter control systems and related methods described herein advantageously may overcome one or more of the above-described limitations associated with use of existing techniques for identifying and changing or maintaining a position and/or orientation of a catheter of a medical device within a subject's body. As discussed herein, the catheter control system may allow a position and/or attitude of a catheter to be precisely and accurately detected and changed or maintained in real-time, thereby reducing incidence of complications arising from undesired migration of the catheter. In particular, the sensor of the catheter control system may provide an accurate means for determining and tracking the catheter position and/or orientation, while the actuators associated with respective openings of the containment structure may provide a compact, responsive means for repositioning and/or reorienting the catheter to, or at least toward, the target position and/or orientation within the subject's body. In this manner, use of the catheter control systems for position or attitude (or combination of both position and attitude) control may avoid the need for repeated use of x-rays or echocardiography and manual repositioning and/or reorienting of the catheter. Further, because the catheter control systems may operate in an autonomous manner, they may reduce the need for physician-patient interaction related to positioning and/or orienting the catheter.

Although the catheter control systems and related methods provided herein may be described as being particularly useful for certain mechanical circulatory support devices, such as PVADs, it will be appreciated that the use of the catheter control systems and methods is not limited to such devices. To the contrary, the catheter control systems and related methods may be used for various other types of medical devices. Further, the control systems and methods may be used in non-medical applications in which the autonomous repositioning and/or reorienting of a device having an active fluid flow therethrough is desirable.

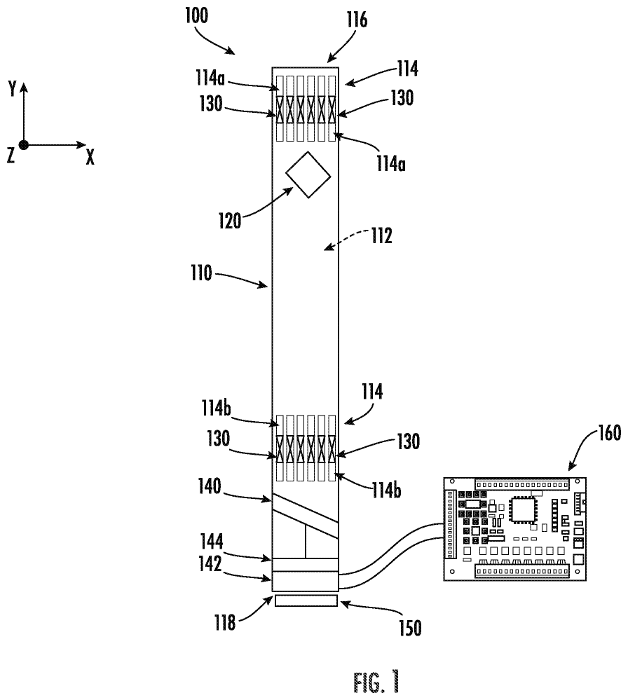

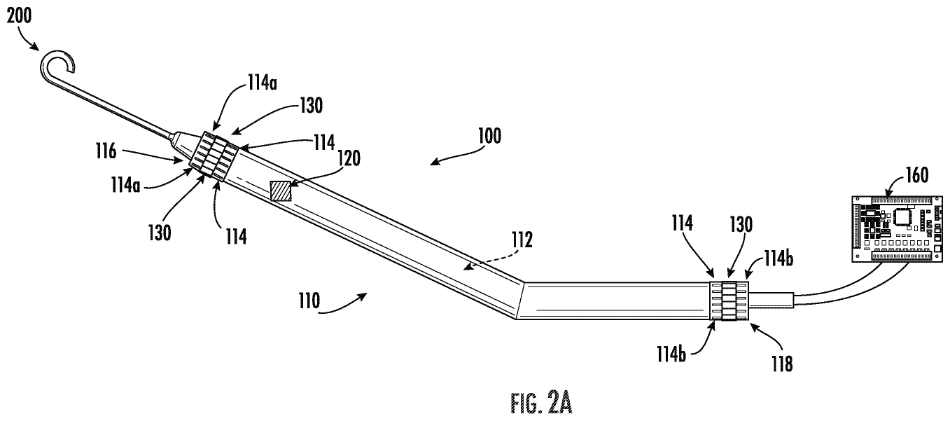

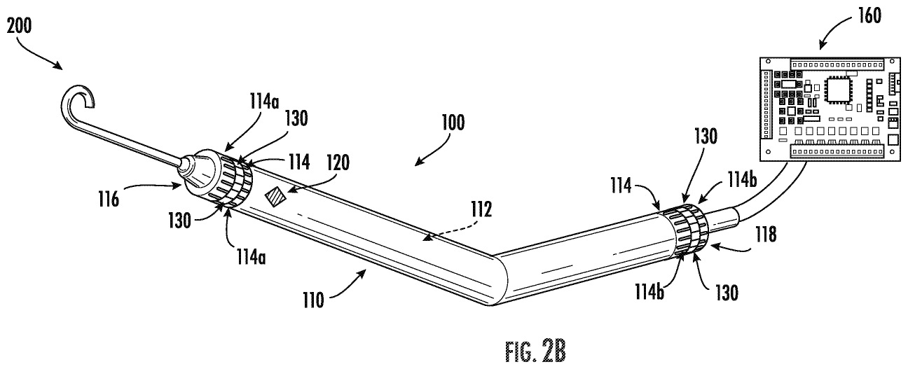

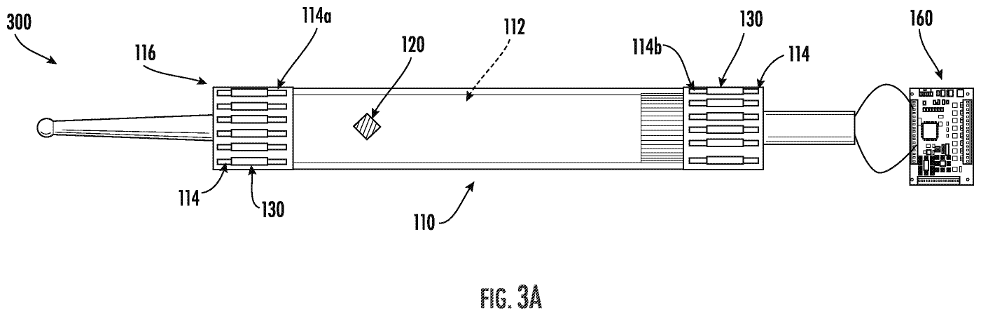

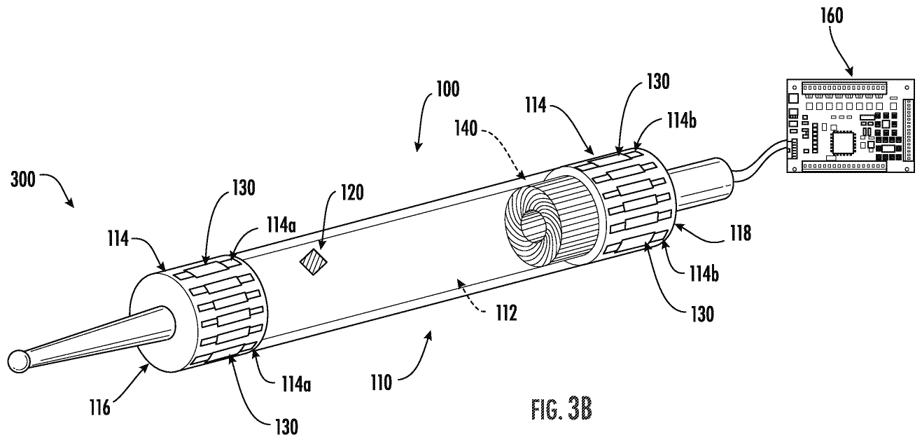

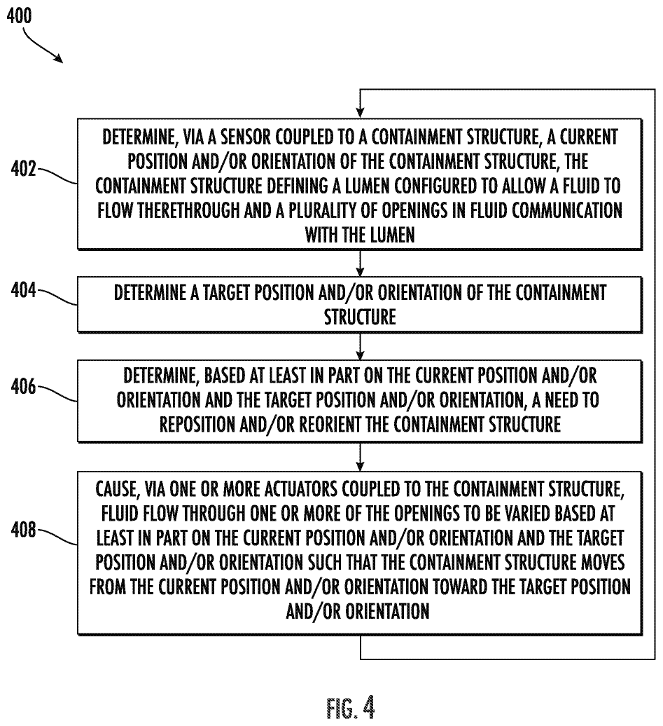

Referring now to FIG. 1, a catheter control system 100 (which also may be referred to as a “position control system,” “an attitude control system”, “a combination of position and attitude control system”, a “control system,” or simply a “system”) in accordance with one or more embodiments of the disclosure is depicted. As described herein, the system 100 may be configured to regulate fluid flow in order to utilize the reactive forces created from the regulated fluid flow to reposition and/or reorient at least a portion of the system 100. Although the system 100 is shown in isolation in FIG. 1, the catheter control system 100 may be attached to or integrated into a medical device, such as a PVAD, as shown in FIGS. 2A, 2B, 3A and 3B. The system 100 may also be used with any device inserted into a body to facilitate tracking, reorienting and repositioning of the device inside the body. In some instances, the system 100 may be used in accordance with the method 400 shown in FIG. 4, although the system 100 may be used in accordance with other methods. As shown in FIG. 1, the system 100 may include a containment structure 110, a sensor 120, a plurality of actuators 130, a pump 140, a power source 150, and a controller 160.

The containment structure 110 (which also may be referred to as a “catheter body”) may define a lumen 112 configured to allow a fluid, such as blood or another bodily fluid, to flow therethrough, and a plurality of openings 114 in fluid communication with the lumen 112. In some embodiments, the containment structure 110 may have a tubular shape with a circular cross-sectional shape. In some embodiments, the containment structure 110 may flexible and configured to be elastically deformed. In other embodiments, the containment structure 110 may be rigid. As shown, the containment structure 110 may have a first end 116 (which also may be referred to as a “distal end” or an “inlet end”) and a second end 118 (which also may be referred to as a “proximal end” or an “outlet end”) positioned opposite one another with respect to a longitudinal extent of the containment structure 110. The lumen 112 may extend from the first end 116 to the second end 118. In some embodiments, the containment structure 110 may include a bellows. In such embodiments, the bellows may be configured to allow a length of the containment structure 110 to be extended or retracted. The bellows may be associated with a bellows actuator configured to extend or retract the bellows and thus the length of the containment structure 110. In some embodiments, each of the openings 114 may have a rectangular shape, although other suitable shapes, such as circular shapes, may be used in other embodiments. In some embodiments, as shown in FIG. 1, the plurality of openings 114 may include a plurality of inlet openings 114a and a plurality of outlet openings 114b. The inlet openings 114a may be positioned closer to the first end 116 of the containment structure 110 and configured to allow fluid to enter the lumen 112. In some embodiments, the inlet openings 114a may be equally spaced apart from one another. The inlet openings 114a may be oriented in a plurality of different directions from one another relative to the containment structure 110 and may be positioned on different surfaces of the containment structure 110. The outlet openings 114b may be positioned closer to the second end 118 of the containment structure 110 and configured to allow fluid to exit the lumen 112. In some embodiments, the outlet openings 114b may be equally spaced apart from one another. In some embodiments, the outlet openings 114b may be oriented in a plurality of different directions from one another relative to the containment structure 110 and may be positioned on different surfaces of the containment structure 110.

Although the containment structure 110 is depicted defining six inlet openings 114a and six outlet openings 114b, the containment structure 110 may define fewer or more of the inlet openings 114a and the outlet openings 114b in other embodiments. According to various embodiments, the containment structure 110 may define a pair of the inlet openings 114a and a pair of the outlet openings 114b, three of the inlet openings 114a and three of the outlet openings 114b, four of the inlet openings 114a and four of the outlet openings 114b, five of the inlet openings 114a and five of the outlet openings 114b, or any other number of the inlet openings 114a and the outlet openings 114b. In some embodiments, the number of the inlet openings 114a may be equal to the number of the outlet openings 114b. In other embodiments, the number of the inlet openings 114a may be greater than or less than the number of the outlet openings 114b. In some embodiments, the plurality of inlet openings 114a may include one or more pairs of the inlet openings 114a oriented in opposite directions. For example, a first inlet opening 114a may be oriented in the +X direction (according to the coordinates depicted in FIG. 1), and a second inlet opening 114a may be oriented in the −X direction. Further, a third inlet opening 114a may be oriented in the +Z direction, a fourth inlet opening 114a may be oriented in the −Z direction, and a fifth inlet opening 114a may be oriented in the +Y direction. In some embodiments, the plurality of outlet openings 114b may include one or more pairs of the outlet openings 114b oriented in opposite directions. For example, a first outlet opening 114b may be oriented in the +X direction, and a second outlet opening 114b may be oriented in the −X direction. Further, a third outlet opening 114b may be oriented in the +Z direction, a fourth outlet opening 114b may be oriented in the −Z direction, and a fifth outlet opening 114b may be oriented in the −Y direction. Various arrangements of the inlet openings 114a and the outlet openings 114b may be used. As discussed herein, fluid flow through the openings 114 may be regulated to result in a desired propulsive thrust. Accordingly, the orientations of the openings may facilitate orientation of the resulting propulsive thrust such that the containment structure 110 may be moved from a current position and/or orientation toward or to the target position and/or orientation. In some embodiments, the containment structure 110 may be formed of any biocompatible materials suitable for allowing the containment structure 110 to elastically flex. In other words, the containment structure 110 may be configured to be elastically deformed from a natural configuration, such as a linear configuration depicted in FIG. 1, upon application of external forces to the containment structure 110 and may tend to return toward or to the natural configuration upon removal of external forces.

The sensor 120 may be coupled to the containment structure 110 and may be configured to detect a current position and/or orientation of the containment structure 110. In some embodiments, as shown, the sensor 120 may be positioned closer to the first end 116 than the second end 118 of the containment structure 110. In other embodiments, the sensor 120 may be positioned closer to the second end 118 than the first end 116 of the containment structure 110, at or near a midpoint of the containment structure 110 between the first end 116 and the second end 118, or anywhere on the containment structure 110 such that the sensor 120 (or an array of sensors) is able to obtain accurate position and/or attitude data. In some applications, positioning the sensor 120 near a portion of the containment structure 110 that is expected to experience a greater degree of movement, such as the first end 116 or the second end 118 in different applications, may enhance sensitivity of the sensor 120. In some embodiments, the sensor 120 may be positioned on an internal surface or an external surface of the containment structure 110. In other embodiments, the sensor 120 may be integrated into the containment structure 110. In other words, the sensor 120 may be positioned between the internal surface and the external surface of the containment structure 110. According to different embodiments, the sensor 120 may include one or more of an accelerometer, a gyroscope, an array of capacitive sensors, an inductive sensor, a magnetic sensor, a piezoelectric sensor, an optical sensor, or any combination thereof.

Each of the actuators 130 may be associated with at least one respective opening 114 of the plurality of openings 114 and may be configured to regulate fluid flow through the at least one respective opening 114. As shown, each of the actuators 130 may be associated with one of the inlet openings 114a or one of the outlet openings 114b. In some embodiments, each of the actuators 130 may be configured to transition between a fully open state, a partially open state, and a closed state for regulating fluid flow through the at least one respective opening 114. In this manner, one or more of the actuators 130 may be caused to assume the open state, the closed state, or an intermediate state between the open state and the closed state (i.e., a partially-open or partially-closed state) for regulating fluid flow through the respective opening 114. In some embodiments, one or more, or all, of the actuators 130 may be associated with two or more of the openings 114. According to different embodiments, the plurality of actuators 130 may include a plurality of valves, a plurality of diaphragms, a plurality of venturi devices, or any combination thereof. As described below, the actuators 130 may be controlled by the electronic controller 160 based on sensor signals received from the sensor 120.

The pump 140 may be configured to induce fluid flow through the lumen 112 of the containment structure 110. In particular, the pump 140 may be configured to create a pressure differential for inducing fluid flow within the lumen 112, thereby causing fluid to enter the inlet openings 114a and exit the outlet openings 114b. In some embodiments, the pump 140 may be positioned at least partially, or entirely, within the containment structure 110. In other embodiments, the pump 140 may be positioned entirely outside of the containment structure 110. In some such embodiments, the pump 140 may be an external pump that is positioned outside of the subject's body during use of the system 100. In some embodiments, as shown, the pump 140 may be a screw pump. In other embodiments, the pump 140 may be a syringe pump, a positive displacement pump, or any other type of pump suitable for inducing fluid flow through the lumen 112. The pump 140 may be associated with a pump actuator 142 that is configured to drive the pump 140. In some embodiments, the pump actuator 142 may be positioned at least partially, or entirely, within the containment structure 110. In other embodiments, the pump actuator 142 may be positioned entirely outside of the containment structure 110. In some embodiments, as shown in FIG. 1, the pump actuator 142 may be, or may include, a pump motor that is coupled to the pump 140 by a gear reducer 144. The gear reducer 144 may be configured to convert the rotational speed of the pump motor of the pump actuator 142 to a rotational speed optimal for driving the pump 140. In other embodiments, the pump actuator 142 may be, or may include, a thermally driven actuator or a piezoelectric actuator.

The power source 150 may be configured to power one or more electronic components of the system 100, such as one or more, or all, of the sensor 120, the actuators 130, pump 140, the pump actuator 142, and the controller 160. In some embodiments, the power source 150 may be positioned at least partially, or entirely, within the containment structure 110. In other embodiments, the power source 150 may be positioned entirely outside of the containment structure 110. The power source 150 may include one or more batteries or any other power storage device suitable for powering the electronic components of the system 100.

The controller 160 may be in operable communication with the sensor 120 and each of the actuators 130. In some embodiments, the controller 160 also may be in operable communication with the pump 140 and/or the pump actuator 142 and the power source 150. In some embodiments, the controller 160 may carry out the method 200 shown in FIG. 4 and described below. The controller 160 may be configured to receive, from the sensor 120, sensor signals indicative of position data and/or attitude data corresponding to a position and/or orientation of the containment structure 110 and, based at least in part on that position data and/or attitude data, cause one or more of the actuators 130 to vary fluid flow through one or more of the openings 114. In some embodiments, the controller 160 may be, or may include, an electronic controller. In some embodiments, the controller 160 may be, or may include, a mechanical controller, a thermal controller, a piezoelectric controller, an acoustic controller, or any other type of controller suitable for controlling the actuators 130. In some embodiments, the controller 160 may include a memory that is configured to store previous position data and/or attitude data obtained via sensor signals received from the sensor 120 over a period of time and compare the previous position data and/or attitude data to current position data and/or attitude data. In some embodiments, the controller 160 may be configured to vary fluid flow through one of more of the openings 114 proportional to a difference between a current position and/or orientation and a target position and/or orientation of the containment structure 110. In some embodiments, the controller 160 may be configured to cause one or more of the actuators 130 to transition between a fully open state, a partially open state, and a closed state for regulating fluid flow. In some embodiments, the controller 160 may be configured to cause one or more of the actuators 130 to vary fluid flow through one or more of the openings 114 based at least in part on a proportional function, a differential function, an integral function, a derivative function, or a combination thereof. In some embodiments, the controller 160 may be configured to cause one or more of the actuators 130 to vary fluid flow through one or more of the openings 114 based at least in part on fuzzy control, a machine learning algorithm or a neural network learning algorithm. In some embodiments, the controller 160 may be or may include a DAQ (Data Acquisition). In some embodiments, the controller 160 may be or may include controller board (such as an Arduino board). Various suitable hardware and software configurations of the controller 160 may be used for carrying out the functionality described herein. In some embodiments, the functionality of the controller 160 described herein may be carried out by an existing controller of a medical device, such as a PVAD, with which the system 100 is associated. In other words, the controller of the medical device may be in operable communication with the respective components of the system 100 and configured to interact with the components in the manner described with respect to the controller 160.

FIGS. 2A and 2B illustrate use of the catheter control system 100 with an example PVAD 200 in accordance with one or more embodiments of the disclosure. FIGS. 3A and 3B illustrate use of the catheter control system 100 with another example PVAD 300 in accordance with one or more embodiments of the disclosure. It will be appreciated that such uses of the system 100 are merely examples, and that the system 100 may be used with various other types of medical devices in other embodiments. In some embodiments, the system 100 may be attached to the PVAD 200, 300. In other embodiments, the system 100 may be integrated into the PVAD 200, 300. In some embodiments, the system 100 may utilize an existing pump of the PVAD 200, 300 to create a pressure differential that induces fluid flow into the inlet openings 114a, through the lumen 112, and out of the outlet openings 114b. In some embodiments, the pump of the PVAD 200, 300 may be in operable communication with the electronic controller 160. During use of the PVAD 200, 300 for treatment of a subject, the controller 160 may operate to cause one or more of the actuators 130 to vary fluid flow through the containment structure 110 based at least in part on a difference existing between a current position and/or orientation and a target position and/or orientation of the containment structure 110 associated with the PVAD 200, 300.

FIG. 4 illustrates a method 400 for controlling a position and/or orientation of a catheter within a subject's body in accordance with one or more embodiments of the disclosure. The method 400 may be implemented by the controller 160 during use of the catheter control system 100. In some embodiments, the electronic controller 160 may include at least one processor coupled to at least one memory that stores computer-executable instructions, with the at least one processor being configured to access the at least one memory and execute the computer-executable instructions for performing the various functions described herein.

At step 402, a current position and/or orientation of the containment structure 110 may be determined via the sensor 120. For example, the controller 160 may receive, from the sensor 120, a sensor signal (which also may be referred to as an “output signal” of the sensor 120) that is indicative of position data and/or attitude data corresponding to the current position and/or orientation of the containment structure 110, and the controller 160 may determine the current position and/or orientation of the containment structure 110 based at least in part on the sensor signal. In some embodiments, the sensor signal (from a single sensor or an array of sensors) may be indicative of one or more of: linear acceleration of the containment structure 110, angular acceleration of the containment structure 110, linear velocity of the containment structure 110, and angular velocity of the containment structure 110, or a combination thereof. In some embodiments, the controller 160 may integrate the sensor signal as a function of time to determine the current position and/or orientation of the containment structure 110. The current position and/or orientation of the containment structure 110 may be determined with respect to a specified datum or reference place. In some embodiments, the current position and/or orientation of the containment structure 110 in three-dimensional space may be determined with respect to three Cartesian coordinates (e.g., the Cartesian coordinates depicted in FIG. 1). In some embodiments, the sensor 120 may continuously detect one or more parameters associated with the position data and/or attitude data for the containment structure 110, and the controller 160 may continuously receive sensor signals indicative of the one or more parameters from the sensor 120. In other embodiments, the sensor 120 may periodically detect one or more parameters associated with the position data and/or attitude data for the containment structure 110 at timed intervals, such as intervals set by a user of the system 100 (e.g., a physician), and the controller 160 may periodically receive sensor signals indicative of the one or more parameters from the sensor 120 at the timed intervals. In some embodiments, the controller 160 may store the position data and/or attitude data for the containment structure 110 over time, for example, at a memory or data storage of or associated with the controller 160. As described above, the sensor 120 may be coupled to the containment structure 110, and the containment structure 110 may define the lumen 112 configured to allow a fluid to flow therethrough and the plurality of openings 114 in fluid communication with the lumen 112.

At step 404, a target position and/or orientation of the containment structure 110 may be determined. Similar to the current position and/or orientation, the target position and/or orientation of the containment structure 110 may be determined with respect to a specified datum or reference place. In some embodiments, the target position and/or orientation of the containment structure 110 in three-dimensional space may be determined with respect to the three Cartesian coordinates. In some embodiments, the target position and/or orientation of the containment structure 110 may be determined based at least in part on a pre-programmed algorithm for identifying an optimal position and/or orientation of the containment structure 110 with respect to the subject's anatomy. In some embodiments, the target position and/or orientation may be determined based at least in part on one or more user inputs received at a user interface of the system 100. For example, the user of the system 100 may interact with the user interface to set the target position and/or orientation for the containment structure 110. In some embodiments, target position data and/or attitude data corresponding to the target position and/or orientation of the containment structure 110 may be stored at a memory or data storage of or associated with the controller 160 and subsequently retrieved by the controller 160 to determine the target position and/or orientation. In such embodiments, the target position data and/or attitude data may be updated by subsequent user interaction with the user interface, and the updated target position data and/or attitude data may be subsequently retrieved by the controller 160 to determine the updated target position and/or orientation.

At step 406, a need to reposition and/or reorient the containment structure 110 may be determined based at least in part on the current position and/or orientation and the target position and/or orientation. In some embodiments, the controller 160 may determine a difference between the current position and/or orientation and the target position and/or orientation. In some embodiments, the controller 160 may determine a difference between the current position and/or orientation and the target position and/or orientation with respect to each of the three Cartesian coordinate directions. In some embodiments, when the difference between the current position and/or orientation and the target position and/or orientation with respect to one or more of the three Cartesian coordinate directions (and/or orientations) exceeds a predetermined threshold value, the controller 160 may determine that a need to reposition and/or reorient the containment structure 110 exists. Conversely, when the difference between the current position and/or orientation and the target position and/or orientation with respect to each of the three Cartesian coordinate directions does not exceed the predetermined threshold value, the controller 160 may determine that a need to reposition and/or reorient the containment structure 110 does not exist. In some embodiments, the controller 160 may use a pre-programmed algorithm to determine whether a need to reposition and/or reorient the containment structure 110 does or does not exist, with the current position and/or orientation and the target position and/or orientation being inputs for the algorithm. When a need to reposition and/or reorient the containment structure 110 does not exist, the controller 160 may return to step 402. When a need to reposition and/or reorient the containment structure 110 does exist, the controller 160 may proceed to step 408.

At step 408, fluid flow through one or more of the openings 114 may be caused to be varied based at least in part on the current position and/or orientation and the target position and/or orientation such that the containment structure 110 moves from the current position and/or orientation toward the target position and/or orientation. In some embodiments, the controller 160 may cause fluid flow through one or more of the openings 114 to be varied via one or more of the actuators 130 coupled to the containment structure 110. For example, the controller 160 may cause one or more of the actuators 130 to transition between an open state and a closed state (or a partially open state) to vary fluid flow through the respective one or more openings 114. In some embodiments, the controller 160 may cause one or more of the actuators 130 to vary fluid flow through the respective one or more openings 114 proportional to a difference between the current position and/or orientation and the target position and/or orientation. In some embodiments, the controller 160 may use a pre-programmed algorithm to determine respective states (i.e., an open state, a closed state, or an intermediate state) of the actuators 130 for varying fluid flow through the respective openings 114 in a desired manner to cause the containment structure 110 to move from the current position and/or orientation toward the target position and/or orientation. Upon determining the respective states of the actuators 130, the controller 160 may send one or more control signals to one or more of the actuators 130 to cause the actuators 130 to vary fluid flow through the respective openings 114 in the desired manner. In some embodiments, the controller 160 also may cause a pressure differential to be created for inducing fluid flow through the lumen 112. For example, the controller 160 may cause the pump 140 to be driven by the pump actuator 142 such that the pump 140 creates a pressure differential for inducing fluid flow through the lumen 112. As described above, the controller 160 may cause the power source 150 to power the pump actuator 142 for driving the pump 140. The pump 140 may induce fluid flow into the inlet openings 114a, through the lumen 112, and out of the outlet openings 114b. Based on the respective states of the actuators 130 associated with the inlet openings 114a and the outlet openings 114b, a propulsive thrust may be generated to move the containment structure 110 from the current position and/or orientation to, or at least toward, the target position and/or orientation. In various embodiments, the controller 160 may modify or maintain the states of any combination of the actuators 130 associated with the inlet openings 114a and the outlet openings 114b to generate a desired propulsive thrust for moving the containment structure 110 from the current position and/or orientation to, or at least toward, the target position and/or orientation. In some embodiments, the containment structure 110 may include a bellows, and a state of the bellows may be modified in conjunction with the respective states of the actuators 130 such that the bellows retracts or extends the containment structure 110 to vary the fluid flow through the lumen 112. In this manner, the containment structure 110 would have a different propulsive thrust effect because of the different length of the containment structure 110 due to the bellows.

After the fluid flow through the one or more of the openings 114 has been varied to cause the containment structure 110 to move from the current position and/or orientation toward the target position and/or orientation at step 408, the method 400 may return to step 402. Accordingly, the method 400 may be an iterative process, with the controller 160 again determining a current position and/or orientation of the containment structure 110, determining a target position and/or orientation of the containment structure 110, determining, based at least in part on the current position and/or orientation and the target position and/or orientation, whether a need to reposition the containment structure 110 does or does not exist, and if so, causing fluid flow through one or more of the openings 114 to be varied based at least in part on the current position and/or orientation and the target position and/or orientation such that the containment structure moves from the current position and/or orientation toward the target position and/or orientation. In this manner, the catheter control system 100 may be used to autonomously control a position and/or orientation of the containment structure 110 within a subject's body.

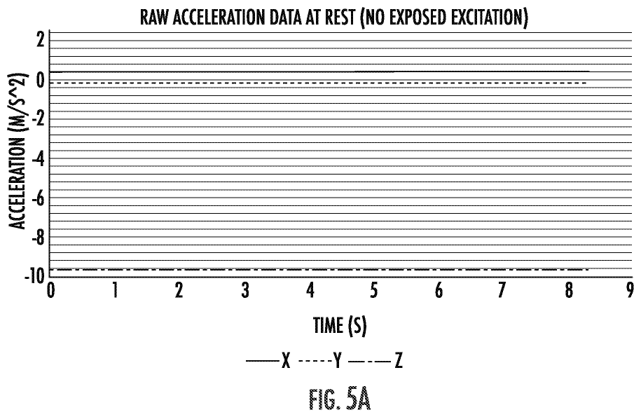

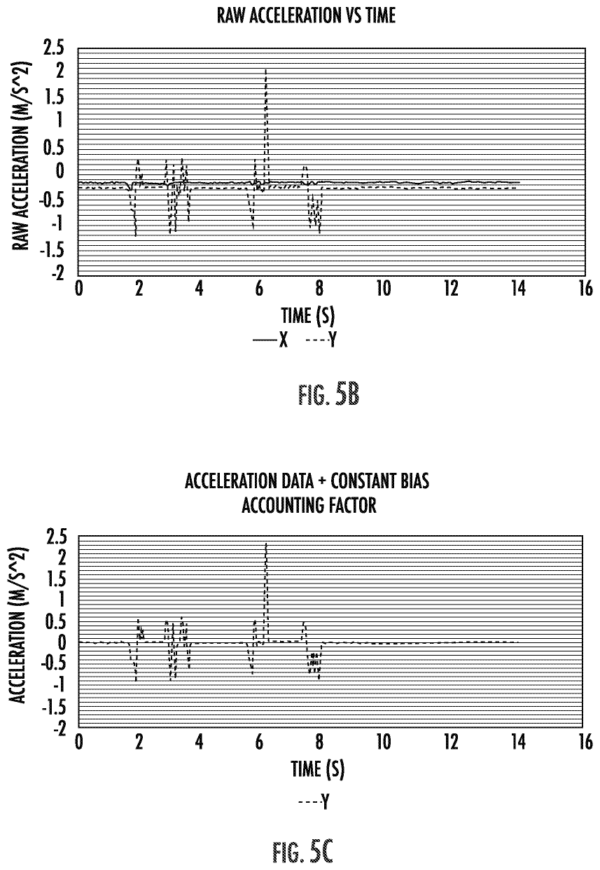

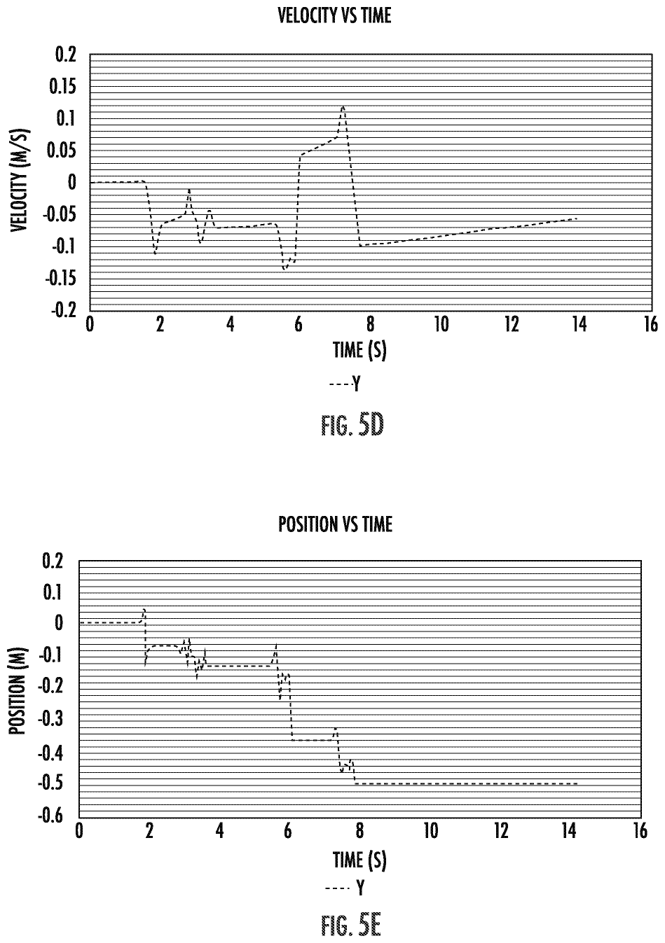

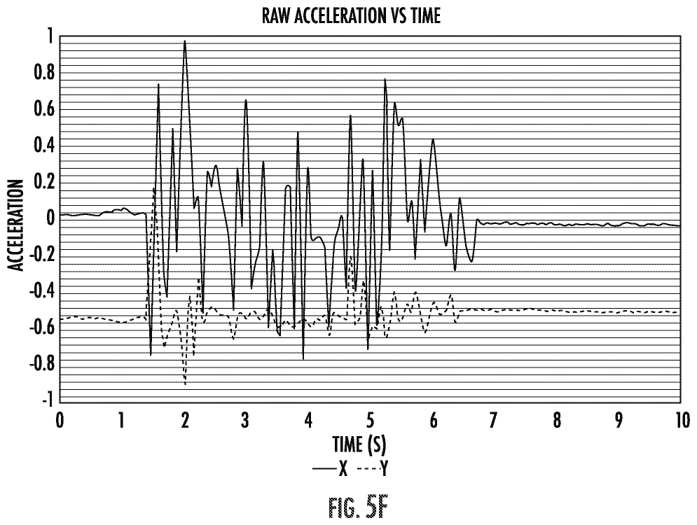

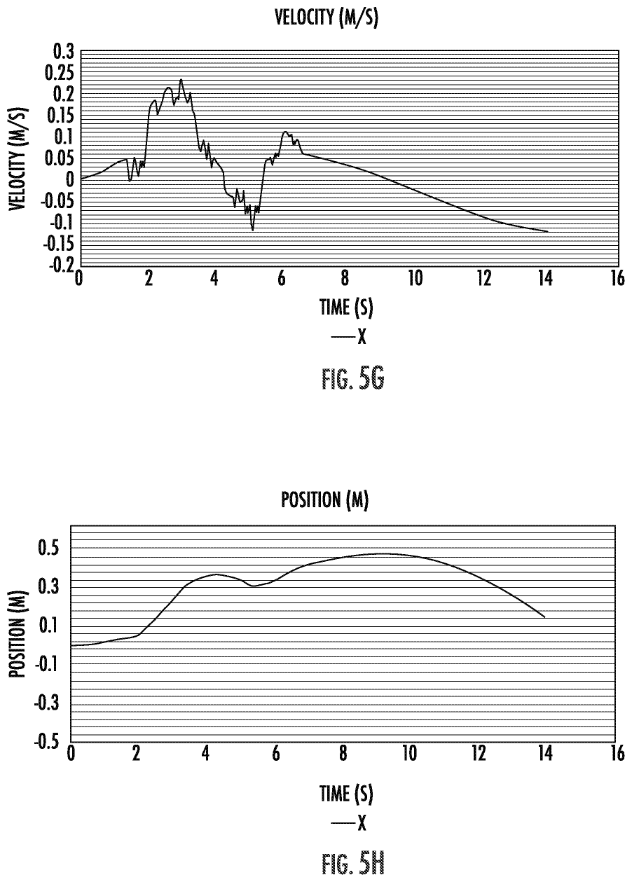

As discussed above, during use of the catheter control system 100, the controller 160 may determine a current position of the containment structure 110. In particular, the controller 160 may receive, from the sensor 120, a sensor signal that is indicative of position data corresponding to the current position of the containment structure 110, and the controller 160 may determine the current position based at least in part on the sensor signal. In some embodiments, the sensor 120 may be or may include an accelerometer, and the sensor signal may be indicative of linear acceleration of the containment structure 110. In some embodiments, the controller 160 may integrate the sensor signal as a function of time to determine the current position of the containment structure 110 in three-dimensional space with respect to three Cartesian coordinates. FIGS. 5A-5H illustrate example data from experimental testing using an accelerometer to determine position.

FIGS. 5A-5C correspond to a first experimental test in which the accelerometer was coupled to an Arduino controller. Initially, baseline trials were conducted, in which the accelerometer was not exposed to excitation or disturbance. In other words, the accelerometer was maintained at rest while acceleration data was collected. FIG. 5A illustrates the raw acceleration as a function of time. As shown, non-zero acceleration was detected along the Z axis due to gravity, while non-zero acceleration also was detected along each of the X axis and the Y axis, although substantially smaller than that detected along the Z axis. The non-zero raw acceleration values signify inherent bias within the accelerometer, which must be accounted for in order to calculate position. Then, the accelerometer was exposed to displacement along a single axis, the Y axis, while acceleration was detected. FIG. 5B illustrates the raw acceleration as a function of time. As shown, inherent bias in the raw acceleration data was observed. Specifically, under resting conditions, the detected acceleration was non-zero. The bias was accounted for by using the average of the resting condition value of acceleration. FIG. 5C illustrates a sum of the raw acceleration and a constant bias accounting factor as a function of time.

FIGS. 5D and 5E correspond to a second experimental test in which the accelerometer was exposed to different displacements along a single axis, the Y axis, in sequence. Specifically, the accelerometer was exposed to a first displacement of 0.0254 meters (1 inch) in the +Y axis direction, a second displacement of 0.0254 meters (1 inch) in the −Y axis direction, a third displacement of 0.0508 meters (2 inches) in the +Y axis direction, and a second displacement of 0.0508 meters (2 inches) in the −Y axis direction. In other words, the accelerometer was exposed to true displacements of one oscillation of 0.0254 meters and another oscillation of 0.0508 meters, such that the true ending position was the same as the true starting position. Raw acceleration data was collected via the accelerometer and used to calculate velocity and position. FIG. 5D illustrates the calculated velocity as a function of time, while FIG. 5E illustrates the calculated position as a function of time. As shown, the calculated position does not reflect the true position due to bias, indicating a need to use a low pass filter and to perform a calibration experiment.

FIG. 5F corresponds to a similar third experimental test in which the accelerometer was exposed to displacements along the X axis instead of the Y axis. FIG. 5F illustrates the raw acceleration as a function of time. Again, a resting condition bias was observed. However, for this experiment, the resting condition bias was not constant, as different bias values were observed prior to the exposed displacement as compared to after the exposed displacement, indicating a need to use a low pass filter.

FIGS. 5G and 5H correspond to a fourth experimental test in which the accelerometer was exposed to different displacements along a single axis, the Y axis, in sequence. Specifically, the accelerometer was exposed to a first displacement of 0.0889 meters (3.5 inches) in the +Y axis direction, a second displacement of 0.0889 meters (3.5 inches) in the −Y axis direction, and a third displacement of 0.0762 meters (3 inches) in the +Y axis direction. Raw acceleration data was collected via the accelerometer and used to calculate velocity and time. FIG. 5G illustrates the calculated velocity as a function of time, while FIG. 5H illustrates the calculated position as a function of time. As shown, a linear drift was observed in the calculated velocity before the exposed displacements (before about 2 seconds) and after the exposed displacements (after about 6 seconds) due to inherent accelerometer bias, and the linear drift can contribute to error in calculating position. Ultimately, significant deviations were observed between the calculated position and the true position, indicating a need to use a low pass filter and to perform a calibration experiment.

In theory, integrating acceleration data twice is sufficient to calculate position. In practice, however, sensor values have inherent uncertainty and bias, as evidenced by the data obtained from the above experiments. When numerically integrating raw acceleration data twice to calculate position, errors can significantly increase. Accordingly, in addition to offset reduction, a filter may be used to process raw data and remove extraneous noise that can compromise further calculations (i.e., integrating acceleration to determine position). In some embodiments, a low pass filter, which may be implemented through MATLAB, may be used to do this.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment. The terms “based at least in part on” and “based on” are synonymous terms which may be used interchangeably herein. The term “and/or” is an inclusive term, covering each of a number of options related by the term as well as the combination of all of the options. For example, the phrase “X and/or Y” is inclusive of option X only, option Y only, and the combination of X and Y.

What is claimed is:

1. A catheter control system comprising:
a containment structure defining:
a lumen configured to allow a fluid to flow therethrough; and
a plurality of openings in fluid communication with the lumen;
a sensor coupled to the containment structure and configured to detect a current position and/or orientation of the containment structure; and
a plurality of actuators coupled to the containment structure, each of the actuators being associated with at least one respective opening of the plurality of openings and configured to regulate fluid flow through the at least one respective opening.

2. The catheter control system of claim 1, wherein the containment structure has a tubular shape.

3. The catheter control system of any one of claim 1 or 2, wherein the containment structure is flexible.

4. The catheter control system of any one of claim 1 or 2, wherein the containment structure is rigid.

5. The catheter control system of any one of claims 1-4, wherein the containment structure has a first end and a second end positioned opposite the first end, and wherein the lumen extends from the first end to the second end.

6. The catheter control system of claim 5, wherein the plurality of openings comprises:
a plurality of inlet openings configured to allow fluid to enter the lumen, each of the inlet openings being positioned closer to the first end than the second end; and
a plurality of outlet openings configured to allow fluid to exit the lumen, each of the outlet openings being positioned closer to the second end than the first end.

7. The catheter control system of claim 6, wherein the inlet openings are positioned at the first end, and wherein the outlet openings are positioned at the second end.

8. The catheter control system of any one of claim 6 or 7, wherein the inlet openings are equally spaced apart from one another, and wherein the outlet openings are equally spaced apart from one another.

9. The catheter control system of any one of claims 6-8, wherein the inlet openings are oriented in a plurality of different directions from one another relative to the containment structure, and wherein the outlet openings are oriented in a plurality of different directions from one another relative to the containment structure.

10. The catheter control system of any one of claims 6-9, wherein the plurality of inlet openings comprises a pair of inlet openings oriented in opposite directions from one another relative to the containment structure, and wherein the plurality of outlet openings comprises a pair of outlet openings oriented in opposite directions from one another relative to the containment structure.

11. The catheter control system of any one of claims 6-9, wherein the plurality of inlet openings comprises at least two inlet openings, and wherein the plurality of outlet openings comprises at least two outlet openings.

12. The catheter control system of any one of claims 6-11, wherein the plurality of inlet openings comprises at least three inlet openings, and wherein the plurality of outlet openings comprises at least three outlet openings.

13. The catheter control system of any one of claims 6-12, wherein the plurality of inlet openings comprises at least six inlet openings, and wherein the plurality of outlet openings comprises at least six outlet openings.

14. The catheter control system of any one of claims 6-13, wherein a number of the inlet openings is equal to a number of the outlet openings.

15. The catheter control system of any one of claims 6-13, wherein a number of the inlet openings is greater than a number of the outlet openings.

16. The catheter control system of any one of claims 6-13, wherein a number of the inlet openings is less than a number of the outlet openings.

17. The catheter control system of any one of claims 1-16, wherein the containment structure comprises a bellows configured to allow a length of the containment structure to be extended or retracted.

18. The catheter control system of claim 17, further comprising a bellows actuator configured to extend or retract the bellows.

19. The catheter control system of any one of claims 1-18, wherein the sensor comprises an accelerometer.

20. The catheter control system of any one of claims 1-19, wherein the sensor comprises a gyroscope.

21. The catheter control system of any one of claims 1-20, wherein the sensor comprises an array of capacitive sensors.

22. The catheter control system of any one of claims 1-21, wherein the sensor comprises an inductive sensor.

23. The catheter control system of any one of claims 1-22, wherein the sensor comprises a magnetic sensor.

24. The catheter control system of any one of claims 1-23, wherein the sensor comprises an optical sensor.

25. The catheter control system of any one of claims 1-24, wherein the sensor comprises a piezoelectric sensor.

26. The catheter control system of any one of claims 1-25, wherein the plurality of openings comprises an inlet opening configured to allow fluid to enter the lumen and an outlet opening configured to allow fluid to exit the lumen, and wherein the sensor is positioned closer to the inlet opening than the outlet opening.

27. The catheter control system of any one of claims 1-26, wherein the sensor is positioned on an external surface of the containment structure.

28. The catheter control system of any one of claims 1-26, wherein the sensor is positioned on an internal surface of the containment structure.

29. The catheter control system of any one of claims 1-28, wherein each of the actuators is configured to transition between a fully open state, a partially open state, and a fully closed state for regulating fluid flow through the at least one respective opening.

30. The catheter control system of any one of claims 1-29, wherein the plurality of actuators comprises a plurality of valves.

31. The catheter control system of any one of claims 1-29, wherein the plurality of actuators comprises a plurality of diaphragms.

32. The catheter control system of any one of claims 1-29, wherein the plurality of actuators comprises a plurality of venturi devices.

33. The catheter control system of any one of claims 1-32, further comprising a pump in fluid communication with the lumen.

34. The catheter control system of claim 33, wherein the pump is configured to create a pressure differential for inducing fluid flow through the lumen.

35. The catheter control system of any one of claim 33 or 34, wherein the pump is positioned at least partially within the containment structure.

36. The catheter control system of any one of claims 33-35, wherein the pump is a screw pump.

37. The catheter control system of any one of claims 33-36, further comprising a pump actuator configured to drive the pump.

38. The catheter control system of claim 37, wherein the pump actuator comprises a pump motor.

39. The catheter control system of claim 37, wherein the pump actuator comprises a thermally driven actuator.

40. The catheter control system of claim 37, wherein the pump actuator comprises a piezoelectric actuator.

41. The catheter control system of any one of claims 37-40, wherein the pump actuator is positioned at least partially within the containment structure.

42. The catheter control system of any one of claims 37-41, further comprising a power source configured to power the pump actuator.

43. The catheter control system of claim 42, wherein the power source is positioned at least partially within the containment structure.

44. The catheter control system of any one of claim 42 or 43, wherein the power source comprises a battery.

45. The catheter control system of any one of claims 1-44, further comprising an electronic controller in operable communication with the sensor and the actuators.

46. The catheter control system of claim 45, wherein the electronic controller is configured to cause one or more of the actuators to vary fluid flow through one or more of the openings based at least in part on the current position and/or orientation and a target position and/or orientation of the containment structure.

47. The catheter control system of claim 46, wherein the electronic controller is configured to cause one or more of the actuators to vary fluid flow through one or more of the openings proportional to a difference between the current position and/or orientation and the target position and/or orientation.

48. The catheter control system of claim 46, wherein the electronic controller is configured to cause one or more of the actuators to vary fluid flow through one or more of the openings based at least in part on a proportional function, a differential function, an integral function, a derivative function, or a combination thereof.

49. The catheter control system of claim 46, wherein the electronic controller is configured to cause one or more of the actuators to vary fluid flow through one or more of the openings based at least in part on a machine learning algorithm or a neural network learning algorithm.

50. The catheter control system of any one of claims 45-49, wherein the electronic controller is configured to cause each of the actuators to transition between a fully open state, a partially open state, and a closed state for regulating fluid flow through the at least one respective opening.

51. A catheter control system comprising:
a containment structure defining:
a lumen configured to allow a fluid to flow therethrough; and
a plurality of openings in fluid communication with the lumen;
a sensor coupled to the containment structure and configured to detect a current position and/or orientation of the containment structure;
a plurality of actuators coupled to the containment structure, each of the actuators being associated with at least one respective opening of the plurality of openings and configured to regulate fluid flow through the at least one respective opening; and
a controller in operable communication with the sensor and the actuators, the controller configured to cause one or more of the actuators to vary fluid flow through one or more of the openings based at least in part on the current position and/or orientation and a target position and/or orientation of the containment structure.

52. The catheter control system of claim 51, wherein the containment structure has a tubular shape.

53. The catheter control system of any one of claim 51 or 52, wherein the containment structure is flexible.

54. The catheter control system of any one of claim 51 or 52, wherein the containment structure is rigid.

55. The catheter control system of any one of claims 51-54, wherein the containment structure has a first end and a second end positioned opposite the first end, and wherein the lumen extends from the first end to the second end.

56. The catheter control system of claim 55, wherein the plurality of openings comprises:
a plurality of inlet openings configured to allow fluid to enter the lumen, each of the inlet openings being positioned closer to the first end than the second end; and
a plurality of outlet openings configured to allow fluid to exit the lumen, each of the outlet openings being positioned closer to the second end than the first end.

57. The catheter control system of claim 56, wherein the inlet openings are positioned at the first end, and wherein the outlet openings are positioned at the second end.

58. The catheter control system of any one of claim 56 or 57, wherein the inlet openings are equally spaced apart from one another, and wherein the outlet openings are equally spaced apart from one another.

59. The catheter control system of any one of claims 56-58, wherein the inlet openings are oriented in a plurality of different directions from one another relative to the containment structure, and wherein the outlet openings are oriented in a plurality of different directions from one another relative to the containment structure.

60. The catheter control system of any one of claims 56-59, wherein the plurality of inlet openings comprises a pair of inlet openings oriented in opposite directions from one another relative to the containment structure, and wherein the plurality of outlet openings comprises a pair of outlet openings oriented in opposite directions from one another relative to the containment structure.

61. The catheter control system of any one of claims 56-59, wherein the plurality of inlet openings comprises at least two inlet openings, and wherein the plurality of outlet openings comprises at least two outlet openings.

62. The catheter control system of any one of claims 56-61, wherein the plurality of inlet openings comprises at least three inlet openings, and wherein the plurality of outlet openings comprises at least three outlet openings.

63. The catheter control system of any one of claims 56-62, wherein the plurality of inlet openings comprises at least six inlet openings, and wherein the plurality of outlet openings comprises at least six outlet openings.

64. The catheter control system of any one of claims 56-63, wherein a number of the inlet openings is equal to a number of the outlet openings.

65. The catheter control system of any one of claims 56-63, wherein a number of the inlet openings is greater than a number of the outlet openings.

66. The catheter control system of any one of claims 56-63, wherein a number of the inlet openings is less than a number of the outlet openings.

67. The catheter control system of any one of claims 51-66, wherein the containment structure comprises a bellows configured to allow a length of the containment structure to be extended or retracted.

68. The catheter control system of claim 67, further comprising a bellows actuator configured to extend or retract the bellows.

69. The catheter control system of any one of claims 61-68, wherein the sensor comprises an accelerometer.

70. The catheter control system of any one of claims 61-69, wherein the sensor comprises a gyroscope.

71. The catheter control system of any one of claims 51-70, wherein the sensor comprises an array of capacitive sensors.

72. The catheter control system of any one of claims 51-71, wherein the sensor comprises an inductive sensor.

73. The catheter control system of any one of claims 51-72, wherein the sensor comprises a magnetic sensor.

74. The catheter control system of any one of claims 51-73, wherein the sensor comprises an optical sensor.

75. The catheter control system of any one of claims 51-74, wherein the sensor comprises a piezoelectric sensor.

76. The catheter control system of any one of claims 51-75, wherein the plurality of openings comprises an inlet opening configured to allow fluid to enter the lumen and an outlet opening configured to allow fluid to exit the lumen, and wherein the sensor is positioned closer to the inlet opening than the outlet opening.

77. The catheter control system of any one of claims 51-76, wherein the sensor is positioned on an external surface of the containment structure.

78. The catheter control system of any one of claims 51-76, wherein the sensor is positioned on an internal surface of the containment structure.

79. The catheter control system of any one of claims 51-78, wherein each of the actuators is configured to transition between a fully open state, a partially open state, and a closed state for regulating fluid flow through the at least one respective opening.

80. The catheter control system of any one of claims 51-79, wherein the plurality of actuators comprises a plurality of valves.

81. The catheter control system of any one of claims 51-79, wherein the plurality of actuators comprises a plurality of diaphragms.

82. The catheter control system of any one of claims 51-79, wherein the plurality of actuators comprises a plurality of venturi devices.

83. The catheter control system of any one of claims 51-82, further comprising a pump in fluid communication with the lumen.

84. The catheter control system of claim 83, wherein the pump is configured to create a pressure differential for inducing fluid flow through the lumen.

85. The catheter control system of any one of claim 83 or 84, wherein the pump is positioned at least partially within the containment structure.

86. The catheter control system of any one of claims 83-85, wherein the pump is a screw pump.

87. The catheter control system of any one of claims 83-86, further comprising a pump actuator configured to drive the pump.

88. The catheter control system of claim 87, wherein the pump actuator comprises a pump motor.

89. The catheter control system of claim 87, wherein the pump actuator comprises a thermally driven actuator.

90. The catheter control system of claim 87, wherein the pump actuator comprises a piezoelectric actuator.

91. The catheter control system of any one of claims 87-90, wherein the pump actuator is positioned at least partially within the containment structure.

92. The catheter control system of any one of claims 87-91, further comprising a power source configured to power the pump actuator.

93. The catheter control system of claim 92, wherein the power source is positioned at least partially within the containment structure.

94. The catheter control system of any one of claim 92 or 93, wherein the power source comprises a battery.

95. The catheter control system of any one of claims 51-94, wherein the controller is configured to cause one or more of the actuators to vary fluid flow through one or more of the openings proportional to a difference between the current position and/or orientation and the target position and/or orientation.

96. The catheter control system of any one of claims 51-95, wherein the controller is configured to cause one or more of the actuators to vary fluid flow through one or more of the openings based at least in part on a proportional function, a differential function, an integral function, a derivative function, or a combination thereof.

97. The catheter control system of any one of claims 51-96, wherein the controller is configured to cause one or more of the actuators to vary fluid flow through one or more of the openings based at least in part on a machine learning algorithm or a neural network learning algorithm.

98. The catheter control system of any one of claims 51-97, wherein the controller is configured to cause each of the actuators to transition between a fully open state, a partially open state, and a closed state for regulating fluid flow through the at least one respective opening.

99. The catheter control system of any one of claims 51-98, wherein the controller comprises an electronic controller.

100. The catheter control system of any one of claims 51-98, wherein the controller comprises a mechanical controller.

101. The catheter control system of any one of claims 51-98, wherein the controller comprises a thermal controller.

102. The catheter control system of any one of claims 51-98, wherein the controller comprises a piezoelectric controller.

103. The catheter control system of any one of claims 51-98, wherein the controller comprises an acoustic controller.

104. The catheter control system of any one of claims 51-103, wherein the controller comprises a memory configured to store at least one previous position and/or orientation of the containment structure detected by the sensor.

105. A method for controlling a position and/or orientation of a catheter within a subject's body, the catheter having a containment structure defining a lumen and a plurality of openings in fluid communication with the lumen, the method comprising:
determining, via a sensor coupled to the containment structure, a current position and/or orientation of the containment structure; and
causing, via one or more actuators coupled to the containment structure, fluid flow through one or more of the openings to be varied based at least in part on the current position and/or orientation and a target position and/or orientation of the containment structure such that the containment structure moves from the current position and/or orientation toward the target position and/or orientation.

106. The method of claim 105, wherein the containment structure has a tubular shape.

107. The method of any one of claim 105 or 106, wherein the containment structure is flexible.

108. The method of any one of claim 105 or 106, wherein the containment structure is rigid.

109. The method of any one of claims 105-108, wherein the containment structure has a first end and a second end positioned opposite the first end, and wherein the lumen extends from the first end to the second end.

110. The method of claim 109, wherein the plurality of openings comprises:
a plurality of inlet openings configured to allow fluid to enter the lumen, each of the inlet openings being positioned closer to the first end than the second end; and
a plurality of outlet openings configured to allow fluid to exit the lumen, each of the outlet openings being positioned closer to the second end than the first end.

111. The method of claim 110, wherein the inlet openings are positioned at the first end, and wherein the outlet openings are positioned at the second end.

112. The method of any one of claim 110 or 111, wherein the inlet openings are equally spaced apart from one another, and wherein the outlet openings are equally spaced apart from one another.

113. The method of any one of claims 110-112, wherein the inlet openings are oriented in a plurality of different directions from one another relative to the containment structure, and wherein the outlet openings are oriented in a plurality of different directions from one another relative to the containment structure.

114. The method of any one of claims 110-113, wherein the plurality of inlet openings comprises a pair of inlet openings oriented in opposite directions from one another relative to the containment structure, and wherein the plurality of outlet openings comprises a pair of outlet openings oriented in opposite directions from one another relative to the containment structure.

115. The method of any one of claims 110-113, wherein the plurality of inlet openings comprises at least two inlet openings, and wherein the plurality of outlet openings comprises at least two outlet openings.

116. The method of any one of claims 110-115, wherein the plurality of inlet openings comprises at least three inlet openings, and wherein the plurality of outlet openings comprises at least three outlet openings.

117. The method of any one of claims 110-116, wherein the plurality of inlet openings comprises at least six inlet openings, and wherein the plurality of outlet openings comprises at least six outlet openings.

118. The method of any one of claims 110-117, wherein a number of the inlet openings is equal to a number of the outlet openings.

119. The method of any one of claims 110-117, wherein a number of the inlet openings is greater than a number of the outlet openings.

120. The method of any one of claims 110-117, wherein a number of the inlet openings is less than a number of the outlet openings.

121. The method of any one of claims 105-120, wherein the containment structure comprises a bellows configured to allow a length of the containment structure to be extended or retracted.

122. The method of claim 121, further comprising extending or retracting the bellows via a bellows actuator.

123. The method of any one of claims 105-122, wherein the sensor comprises an accelerometer.

124. The method of any one of claims 105-123, wherein the sensor comprises a gyroscope.

125. The method of any one of claims 105-124, wherein the sensor comprises an array of capacitive sensors.

126. The method of any one of claims 105-125, wherein the sensor comprises an inductive sensor.

127. The method of any one of claims 105-126, wherein the sensor comprises a magnetic sensor.

128. The method of any one of claims 105-127, wherein the sensor comprises an optical sensor.

129. The method of any one of claims 105-128, wherein the sensor comprises a piezoelectric sensor.

130. The method of any one of claims 105-129, wherein the plurality of openings comprises an inlet opening configured to allow fluid to enter the lumen and an outlet opening configured to allow fluid to exit the lumen, and wherein the sensor is positioned closer to the inlet opening than the outlet opening.

131. The method of any one of claims 105-130, wherein the sensor is positioned on an external surface of the containment structure.

132. The method of any one of claims 105-130, wherein the sensor is positioned on an internal surface of the containment structure.

133. The method of any one of claims 105-132, wherein determining the current position and/or orientation of the containment structure comprises integrating an output signal of the sensor as a function of time.

134. The method of claim 133, wherein the output signal is indicative of one or more of: (i) linear acceleration of the containment structure, (ii) angular acceleration of the containment structure, (iii) linear velocity of the containment structure, and (iv) angular velocity of the containment structure.

135. The method of any one of claims 105-134, wherein causing fluid flow through the one or more of the openings to be varied comprises causing the one or more of the actuators to transition between a fully open state, a partially open state, and a closed state.

136. The method of any one of claims 105-135, wherein the plurality of actuators comprises a plurality of valves.

137. The method of any one of claims 105-135, wherein the plurality of actuators comprises a plurality of diaphragms.

138. The method of any one of claims 105-135, wherein the plurality of actuators comprises a plurality of venturi devices.

139. The method of any one of claims 105-138, further comprising causing, via a pump, a pressure differential to be created for inducing fluid flow through the lumen.

140. The method of claim 139, wherein the pump is positioned at least partially within the containment structure.

141. The method of any one of claim 139 or 140, wherein the pump is a screw pump.

142. The method of any one of claims 139-141, further comprising causing the pump to be driven via a pump actuator.

143. The method of claim 142, wherein the pump actuator comprises a pump motor.

144. The method of claim 142, wherein the pump actuator comprises a thermally driven actuator.

145. The method of claim 142, wherein the pump actuator comprises a piezoelectric actuator.

146. The method of any one of claims 142-145, wherein the pump actuator is positioned at least partially within the containment structure.

147. The method of any one of claims 142-146, further comprising causing the pump actuator to be powered via a power source.

148. The method of claim 147, wherein the power source is positioned at least partially within the containment structure.

149. The method of any one of claim 147 or 148, wherein the power source comprises a battery.

150. The method of any one of claims 105-149, wherein causing fluid flow through the one or more openings to be varied comprises causing, via an electronic controller, the one or more actuators to vary fluid flow through the one or more of openings proportional to a difference between the current position and/or orientation and the target position and/or orientation.

151. The method of any one of claims 105-149, wherein causing fluid flow through the one or more openings to be varied comprises causing, via an electronic controller, the one or more actuators to vary fluid flow through the one or more of openings based at least in part on a proportional function, a differential function, an integral function, a derivative function, or a combination thereof.

152. The method of any one of claims 105-149, wherein causing fluid flow through the one or more openings to be varied comprises causing, via an electronic controller, the one or more actuators to vary fluid flow through the one or more of openings based at least in part on a machine learning algorithm or a neural network learning algorithm.

153. The method of any one of claims 105-152, wherein causing fluid flow through the one or more openings to be varied comprises causing, via an electronic controller, the one or more actuators to transition between a fully open state, a partially open state and a closed state.
